(19) United States
(12) Patent Application Publication
(10) Pub. No.: US 2004/0185454 A1
(43) Pub. Date: Sep. 23, 2004

(54) Identification and quantification of microbial species in a sample

(76) Inventors: Alison M. Jones, Eden Prairie, MN (US); W. Wade Robey, Excelsior, MN (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/395,009

(22) Filed: Mar. 21, 2003

(51) Int. Cl. C12Q 1/68; C07H 21/04; C12P 19/34

(52) U.S. Cl. 435/6; 435/91.2; 536/25.32

(57) ABSTRACT

In situ hybridization and polypeptide-based methods for using cpn60 to detect and/or quantify microbial organisms within a biological or non-biological sample are provided, as are cpn60 probes and antibodies for use in methods of the invention, and kits containing such probes and antibodies.

### TECHNICAL FIELD

This invention relates to determining microbial profiles, and more particularly to determining microbial profiles based on detection and quantification of chaperonin 60 (cpn60) nucleic acids and polypeptides from various microbial species present within a sample.

### BACKGROUND

Microbial profiles are representations of individual strains, subspecies, species, and/or genera of microorganisms within a community of microorganisms. Generally, determining a microbial profile involves taxonomic and/or phylogenetic identification of the microbes in a community. A microbial profile also can include quantitative information about one or more members of the community. Once one or more microorganisms have been identified in a microbial community, microbial profiles can be presented as, for example, lists of microorganisms, graphical or tabular representations of the presence and/or numbers of microorganisms, or any other appropriate representation of the diversity and/or population levels of the microorganisms in a community. Microbial profiles are useful for identifying pathogenic and non-pathogenic microbial organisms in biological and non-biological samples (e.g., samples from animals, the environment, or inanimate objects).

A microbial profile can be determined using any of a number of methods. For example, the microbes in a sample can be cultured and colonies identified and/or enumerated. It has been estimated, however, that culturing typically recovers only about 0.1% of the microbial species in a sample (based on comparisons between direct microscopic counts and recovered colony-forming units). An improvement on culture-based methods is a community-level physiological profile. Such a profile can be determined by monitoring the capacity of a microbial community to utilize a particular carbon source, with subsequent detection of the end product of metabolism of the carbon source. Profiling the physiology of a microbial community can yield qualitative and semi-quantitative results.

Culture-independent methods to determine microbial profiles can include extracting and analyzing microbial macromolecules from a sample. Useful target molecules typically include those that as a class are found in all microorganisms, but are diverse in their structures and thereby reflect the diversity of the microbes. Examples of target molecules include phospholipid fatty acids (PLFA), polypeptides, and nucleic acids. PLFA analysis is based on the universal presence of modified fatty acids in microbial membranes, and is useful as a taxonomic tool. PLFAs are easily extracted from samples, and separation of the various signature structures reveals the presence and abundance of classes of microbes. This method requires appropriate signature molecules, which often are not known or may not be available for the microbes of interest. In addition, the method requires that an organism's PLFA content does not change under different metabolic conditions. Another limitation to using PLFAs as target molecules is that widely divergent organisms may have the same signature set of PLFAs.

Other less direct measures can be made that can provide insight into changes that might be taking place in the microbial profile within a particular environment. For example, pathogenic changes in the gastrointestinal tract (GIT) microbial profile of an animal may lead to morphometric changes in GIT structure. These morphometric changes can be measured by, for sample, excising GIT tissues and histologically evaluating for the number, size, shape, mucosal-cell turnover, and condition of the villi. The microscopic appearance of the villi can correlate with the microbial ecology of the animal, as many of the resident organisms attach directly to the mucosa and can cause damage and/or destruction of the absorptive surface.

Techniques such as immunohistochemical analysis also can be employed as indicative measures of pathogenic microbes in animal tissues. The presence of circulating leukocytic cytokines (lymphokines and monokines), as well as the presence of immunoglobulins (e.g., IgM, IgQ, or IgA),either in the systemic circulation or localized in a tissue at the site of an antigenic insult can be correlated with the presence of potentially deleterious microbes.

Various nucleic acid-based assays also can be employed to determine a microbial profile. Some nucleic acid-based population methods use, for example denaturation and reannealing kinetics to derive an indirect estimate of the guanine and cytosine (%G+C) content of the DNA in a sample. The %G+C technique provides an overall view of the microbial community, but typically is sensitive only to massive changes in the make-up of the community.

Genetic fingerprinting also can be used to determine a microbial profile. Genetic fingerprinting utilizes random-sequence oligonucleotide primers that hybridize specifically to random sequences throughout the genome. Amplification results in a multitude of products, and the distribution of these products is referred to as a genetic fingerprint. Particular patterns can be associated with a community of microbes in the sample. Genetic fingerprinting, however, lacks the ability to conclusively identify specific microbial species.

Denaturing or temperature gradient gel electrophoresis (DGGE or TGGE) is another technique that can be used to determine a microbial profile. As amplification products are electrophoresed in gradients with increasing denaturant or temperature, the double-stranded molecule melts and its mobility is reduced. The melting behavior is determined by the nucleotide sequence, and unique sequences will resolve into individual bands. Thus, a D/TGGE gel yields a genetic fingerprint characteristic of the microbial community, and the relative intensity of each band reflects the abundance of the corresponding microorganism. An alternative format includes single-stranded conformation polymorphism (SSCP). SSCP relies on the same physical basis as %G+C renaturation methods, but reflects a significant improvement over such methods.

In addition, a microbial profile can be determined using terminal restriction fragment length polymorphism (TRFLP) analysis. Amplification products can be analyzed for the presence of known sequence motifs using restriction endonucleases that recognize and cleave double-stranded nucleic acids at these motifs. For example, the enzyme HhaI cuts at 5′-GCGC-3′ sites. Amplification products can be tagged at one end with a fluorescently labeled primer and digested with HhaI. Resolution of the digest by electrophoresis will yield a series of fluorescent bands with lengths determined by how far a 5′-GCGC-3′ motif lies from the terminal tag. The principal advantages of TRFLP are its robustness and its low cost. Unlike D/TGGE, experimental conditions need not be stringently controlled since the profiles are size-based and thus can be generated by a variety of gel systems, including automated DNA sequencing machines. Alternative approaches include “amplified ribosomal DNA restriction analysis (AADRA)” in which the entire amplification product, rather than just the terminal fragment, is considered. AADRA, however, becomes unmanageable with communities containing many species.

A microbial profile also can be determined by cloning and sequencing microbial nucleic acids present in a biological or non-biological sample. Cloning of individual nucleic acids into Escherichia coli and sequencing each nucleic acid gives the highest density of information but requires the most effort. Although sequencing of nucleic acids is an automated process, routine monitoring of changes in the microbial profile of an animal by cloning and sequencing nucleic acids from the microorganisms still requires considerable time and effort.

Genotyping of 16S ribosomal DNA (rDNA) is another way to determine a microbial profile. 16S rDNA sequences are universal and are composed of both (1) highly conserved regions, which allow for design of common amplification primers, and (2) open reading frame (ORF) regions containing sequence variations, which allow for phylogenetic differentiation. 16S ribosomal sequences are relatively abundant in the RNA form. In addition to amplification using oligonucleotide primers, genotyping of 16S rDNA can be performed using other methods including restriction fragment length polymorphism (RFLP) analysis with Southern blotting.

Despite the existence of numerous methods for determining microbial profiles, a method that is rapid, sensitive, and quantitative would have significant utility.

### SUMMARY

The invention provides cpn60 nucleic acid-based and polypeptide-based methods that can be used to determine a microbial profile of a sample. Methods of the invention are rapid and sensitive, and can be used to detect the presence or absence of cpn60-containing microbes in general, as well as to identify what species of microbes are present and in what amounts. Methods of the invention can include using cpn60 nucleic acid probes to detect and quantify cpn60 nucleic acids by in situ hybridization, for example. Such probes for detecting cpn60-containing microbial species also are provided by the invention, as are kits containing such probes. Methods of the invention also can include detecting and quantifying cpn60 polypeptides using, for example, anti-cpn60 antibodies.

In one aspect, the invention features a method for quantifying the amount of one or more microbial species in a biological or non-biological sample. The method can include (a) contacting the sample in situ with at least one labeled cpn60 probe under conditions wherein the probe preferentially hybridizes to cpn60 nucleic acids, if present, in the sample; and (b) quantifying the amount of probe hybridized to the sample, wherein the amount of hybridized probe is correlated with the amount of the microbial species in the sample. The at least one cpn60 probe can be labeled with a fluorescent moiety (e.g., 7-amino-4-methylcoumarin-3-acetic acid, 5-carboxy-X-rhodamine, 6-carboxy-X-rhodamine, lissamine rhodamine B, 5-carboxyfluorescein, 6-carboxyfluorescein, fluorescein-5-isothiocyanate, 7-diethylaminocoumarin-3-carboxylic acid, tetramethylrhodamine-5-isothiocyanate, tetramethylrhodamine-6-isothiocyanate, 5-carboxytetramethylrhodamine, 6-carboxytetramethylrhodamine, 7-hydroxycoumarin-3-carboxylic acid, 6-[fluorescein 5-carboxamido]hexanoic acid, 6-[fluorescein 6-carboxamido]hexanoic acid, N-(4,4-difluoro-5,7-dimethyl-4-bora-3a ,4a diaza-3-indacenepropionic acid, eosin-5-isothiocyanate, or erythrosin-5-isothiocyanate), and the hybridization can be fluorescent in situ hybridization.

The correlation can employ a standard curve of hybridization to cpn60 nucleic acids from known amounts of microbial species. The sample can be contacted with at least two labeled cpn60 probes. The at least two labeled cpn60 probes can be labeled with different fluorescent moieties. The sample can be selected from the group consisting of a biological tissue, a biological fluid, a biological elimination product, a water sample, a soil sample, and a swab from an inanimate object. The one or more microbial species can belong to genera selected from the group consisting of Escherichia, Salmonella, Campylobacter, Staphylococcus, Clostridium, Pseudomonas, Bifidobacterium, Bacillus, Enterococcus, Acanthamoeba, Cryptosporidium, Tetrahymena, Aspergillus, Candida, and Saccharomyces.

In another aspect, the invention features a method for quantifying the amount of one or more microbial species in a biological or non-biological sample. The method can include detecting and/or quantifying the amount of cpn60 polypeptide from the microbial species, if present, in the sample, wherein the amount of the cpn60 polypeptide is correlated with the amount of the microbial species in the sample. The detecting and/or quantifying can include contacting the sample with an anti-cpn60 antibody. The anti-cpn60 antibody can be detectably labeled. The anti-cpn60 antibody can be a monoclonal antibody or a polyclonal antibody. The detecting and/or quantifying can further include contacting the sample with a second antibody. The second antibody can be an anti-cpn60 antibody, or the second antibody can be an antibody that does not bind to cpn60. The detecting and/or quantifying can include a “sandwich” assay or an enzyme linked immunosorbent assay. The sample can be selected from the group consisting of a biological tissue, a biological fluid, a biological elimination product, a water sample, a soil sample, and a swab from an inanimate object. The one or more microbial species can belong to genera selected from the group consisting of Escherichia, Salmonella, Campylobacter, Staphylococcus, Clostridium, Pseudomonas, Bifidobacterium, Bacillus, Enterococcus, Acanthamoeba, Cryptosporidium, Tetrahymena, Aspergillus, Candida, and Saccharomyces.

In another aspect, the invention features a method for identifying one or more microbial species in a biological or non-biological sample. The method can include detecting cpn60 polypeptides from the one or more microbial species, if present, in the sample. The detecting can include contacting the sample with an anti-cpn60 antibody. The anti-cpn60 antibody can be detectably labeled. The anti-cpn60 antibody can be a monoclonal antibody or a polyclonal antibody. The detecting can further include contacting the sample with a second antibody. The second antibody can be an anti-cpn60 antibody, or the second antibody can be an antibody that does not bind to cpn60. The detecting can include a “sandwich” assay or an enzyme linked immunosorbent assay. The sample can be selected from the group consisting of a biological tissue, a biological fluid, a biological elimination product, a water sample, a soil sample, and a swab from an inanimate object. The one or more microbial species can belong to genera selected from the group consisting of Escherichia, Salmonella, Campylobacter, Staphylococcus, Clostridium, Pseudomonas, Bifidobacterium, Bacillus, Enterococcus, Acanthamoeba, Cryptosporidium, Tetrahymena, Aspergillus, Candida, and Saccharomyces.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DETAILED DESCRIPTION

Detection and quantification of microbial organisms, including quantitative forms of microbial profiles, can be determined using methods that involve detection of cpn60 nucleic acid molecules. Methods of the invention are rapid and sensitive, and can be used to qualitatively and quantitatively detect cpn60-containing microbes. Using cpn60 probes, methods of the invention can include detecting and quantifying cpn60 nucleotide sequences using, for example, FISH. The invention provides probes for detecting cpn60-containing microbial species, as well as methods for using such probes to quantify the amount of one or more microbial species in a sample. The invention also provides kits containing cpn60 probes. In addition, the invention provides methods that include detecting and quantifying cpn60 polypeptides using, for example, anti-cpn60 antibodies, as well as kits containing anti-cpn60 antibodies.

As used herein, “microbes” refer to bacteria, protozoa, and fungi. Microbial communities for which a microbial profile can be generated include, without limitation, prokaryotic genera such as Staphylococcus, Streptococcus, Pseudomonas, Escherichia, Bacillus, Brucella, Chlamydia, Clostridium, Shigella, Mycobacterium, Agrobacterium, Bartonella, Borellia, Bradyrhizobium, Ehrlichia, Haemophilus, Helicobacter, Heliobacter, Lactobacillus, Neisseria, Rhizobium, Streptomyces, Synechococcus, Zymomonas, Synechocyotis, Mycoplasma, Yersinia, Vibrio, Burkholderia, Franciscella, Legionella, Salmonella, Bifidobacterium, Enterococcus, Enterobacter, Citrobacter, Bacteroides, Prevotella, Xanthomonas, Xylella, and Campylobacter; protozoa genera such as Acanthamoeba, Cryptosporidium, and Tetrahymena; and fungal genera such as Aspergillus, Colletrotrichum, Cochtiobolus, Helminthosporium, Microcyclus, Puccinia, Pyricularia, Deuterophoma, Monilia, Candida, and Saccharomyces.

Quantitative information about microbial levels can be obtained from various samples. As used herein, “biological sample” refers to any sample obtained, directly or indirectly, from a subject animal or control animal. Representative biological samples that can be obtained from an animal include or are derived from biological tissues, biological fluids, and biological elimination products (e.g., feces). Biological tissues can include biopsy samples or swabs of the biological tissue of interest, e.g., nasal swabs, throat swabs, or dermal swabs. The tissue can be any appropriate tissue from an animal, such as a human, cow, pig, horse, goat, sheep, dog, cat, bird, monkey, fish, clam, oyster, mussel, lobster, shrimp, and crab. Depending on the microbial organism, the tissue of interest to sample (e.g., by biopsy or swab) can be, for example, an eye, a tongue, a cheek, a hoof, a beak, a snout, a foot, a hand, a mouth, a teat, the gastrointestinal tract, a feather, an ear, a nose, a mucous membrane, a scale, a shell, the fur, or the skin.

Biological fluids can include bodily fluids (e.g., urine, milk, lachrymal fluid, vitreous fluid, sputum, cerebrospinal fluid, sweat, lymph, saliva, semen, blood, or serum or plasma derived from blood); a lavage such as a breast duct lavage, lung lavage, a gastric lavage, a rectal or colonic lavage, or a vaginal lavage; an aspirate such as a nipple or teat aspirate; a fluid such as a cell culture or a supernatant from a cell culture; and a fluid such as a buffer that has been used to obtain or resuspend a sample, e.g., to wash or to wet a swab in a swab sampling procedure. Biological samples can be obtained from an animal using methods and techniques known in the art. See, for example, Diagnostic Molecular Microbiology: Principles and Applications (Persing et al. (eds.), 1993, American Society for Microbiology, Washington D.C).

Biological samples also can be obtained from the environment (e.g., air, water, or soil). Methods are known for extracting biological samples (e.g., cells) from such samples. Additionally, a biological sample suitable for use in the methods of the invention can be a substance that one or more animals have contacted. For example, an aqueous sample from a water bath, a chill tank, a scald tank, or other aqueous environments with which a subject or control animal has been in contact, can be used in the methods of the invention to evaluate a microbial profile. A soil sample that one or more subject or control animals have contacted, or on which an animal has deposited fecal or other biological material, also can be used in the methods of the invention. For example, nucleic acids can be isolated from such biological samples using methods and techniques known in the art. See, for example, Diagnostic Molecular Microbiology: Principles and Applications (supra).

Methods of the present invention also can be used to detect the presence of microbial organisms in or on non-biological samples. For example, a fomite may be sampled to detect the presence or absence of a microbial organism. A fomite is a physical (inanimate) object that serves to transmit, or is capable of transmitting, an infectious agent, e.g., a microbial pathogen, from animal to animal. (It is noted that inanimate objects such as food, air, and liquids are not considered fomites, but are considered infectious “vehicles,” or media that are routinely taken into the body.) Indeed, one study that evaluated the presence of Salmonella spp., Listeria spp., and Yersinia spp. pathogenic microbes on various abbatoir fomites detected Salmonella spp. on 11.1% of meat cleavers, 6.25% of worktables, and 5.6% of floors; Yersinia enterocolitica was found on 16.7% of slaughter floors and on 12.5% of worktables; and Listeria monocytogenes was isolated from 13.3% of cold room floor swabs and on 7.1% of hand-wash basins. See Kathryn Cooper, Guelph Food Technology Centre, “The Plant Environment Counts: Protect your Product through Environmental Sampling,” Meat & Poultry, May 1999. Nonlimiting examples of fomites include utensils, knives, drinking glasses, food processing equipment, cutting surfaces, cutting boards, floors, ceilings, walls, drains, overhead lines, ventilation systems, waste traps, troughs, machines, toys, storage boxes, toilet seats, door handles, clothes, gloves, bedding, combs, shoes, changing tables (e.g., for diapers), diaper bins, toy bins, food preparation tables, food transportation vehicles (e.g., rail cars and shipping vessels), gates, ramps, floor mats, foot pedals of vehicles, sinks, washing facilities, showers, tubs, buffet tables, surgical equipment and instruments, and analytical instruments and equipment.

A microbial organism may be left as a residue on a fomite. In such cases, it is important to detect accurately the presence of the organism on the fomite in order to prevent the spread of the organism. For example, it is known that microbes may exist in viable but nonculturable forms on fomites, or that nonculturable bacteria of selected species can be resuscitated to a culturable state under certain conditions. Often such nonculturable bacteria are present in biofilms on fomites. Accordingly, detection methods that rely on culturable forms may significantly under-report microbial contamination on fomites. The methods of the present invention, including PCR-based methods, can aid in the detection and quantification of microbial organisms, particularly nonculturable forms, by detection of cpn60-specific nucleic acid sequences.

The sample also can be a food sample. For example, the sample may be a prepared food sample, e.g., from a restaurant. Such a prepared food sample may be either cooked or raw (e.g., salads, juices). In other embodiments, the food sample may be unprocessed and/or raw, e.g., a tissue sample of an animal from a slaughterhouse, either prior to or after slaughter. The food sample may be perishable. Typically, food samples will be taken from food products such as beef, pork, poultry, seafood, dairy, fruit, vegetable, seed, nut, fungus, and grain. Dairy food samples include milk, eggs, and cheese.

Methods for collecting and storing biological and non-biological samples are generally known to those of skill in the art. For example, the Association of Analytical Communities International (AOAC International) publishes and validates sampling techniques for testing foods and agricultural products for microbial contamination. See also WO 98/32020 and U.S. Pat. No. 5,624,810, which set forth methods and devices for collecting and concentrating microbes from the air, a liquid, or a surface. WO 98/32020 also provides methods for removing somatic cells, or animal body cells present at varying levels in certain samples.

In particular embodiments of the methods described herein, a separation and/or concentration step may be necessary to separate microbial organisms from other components of a sample or to concentrate the microbes to an amount sufficient for rapid detection. For example, a sample suspected of containing a microbial organism may require a selective enrichment of the organism (e.g., by culturing in appropriate media, e.g., for 6-96 hours or longer) prior to employing the detection methods described herein. Alternatively, appropriate filters and/or immunomagnetic separations can concentrate a microbial pathogen without the need for an extended growth stage. For example, antibodies specific for a cpn60-encoded polypeptide can be attached to magnetic beads and/or particles. Multiplexed separations, in which two or more concentration processes are employed also are contemplated, e.g., centrifugation, membrane filtration, electrophoresis, ion-exchange, affinity chromatography, and immunomagnetic separations.

Certain air or water samples may need to be concentrated. For example, certain air sampling methods require the passage of a prescribed volume of air over a filter to trap any microbial organisms, followed by isolation of the organisms into a buffer or liquid culture. Alternatively, the focused air is passed over a plate (e.g., agar) medium for growth of any microbial organisms.

Methods for sampling a tissue or a fomite with a swab are known to those of skill in the art. Generally, a swab is hydrated (e.g., with an appropriate buffer, such as Cary-Blair medium, Stuart's medium, Amie's medium, PBS, buffered glycerol saline, or water) and used to sample an appropriate surface (a fomite or tissue) for a microbial organism. Any microbe present is then recovered from the swab, such as by centrifugation of the hydrating fluid away from the swab, removal of supernatant, and resuspension of centrifugate in an appropriate buffer, or by washing of the swab with additional diluent or buffer. The recovered sample then may be analyzed according to the methods described herein for the presence of a microbial pathogen. Alternatively, the swab may be used to culture a liquid or plate (e.g., agar) medium in order to promote the growth of any pathogen for later testing. Suitable swabs include both cotton and sponge swabs; see, for example, those provided by Tecra®, such as the Tecra ENVIROSWAB®.

Samples can be processed (e.g., by nucleic acid extraction methods and/or kits known in the art) to release nucleic acid or in some cases, a biological sample can be contacted directly with PCR reaction components and appropriate oligonucleotide primers and probes.

cpn60 Nucleic Acids

The term “nucleic acid” as used herein encompasses both RNA and DNA, including genomic DNA. A nucleic acid can be double-stranded or single-stranded. The choice of target nucleic acid sequence to use for quantifying a microbial organism (e.g., when determining a quantitative microbial profile) depends on whether the sequences provide both broad coverage and discriminatory power. Ideally, the target should be present in all members of a given microbial community and be detectable in each member with equal efficiency using common probes, yet have distinct sequences. cpn60 (also known as hsp60 or GroEL) nucleic acid sequences are particularly useful targets for determining a microbial profile by, for example, hybridization. Chaperonin proteins are molecular chaperones required for proper folding of polypeptides in vivo. cpn60 is found universally in prokaryotes and in the organelles of eukaryotes, and can be used as a species-specific target and/or probe for identification and classification of microorganisms. Sequence diversity of this protein-encoding gene between and within bacterial genera appears greater than that of 16S rDNA sequences, making cpn60 a superior target sequence with more distinguishing power for microbial identification at the species level than 16S rDNA.

The invention provides methods to detect and quantify the amount of cpn60-containing microbial species by in situ hybridization of a cpn60 probe to all or a portion of a cpn60 nucleic acid. Sequences of cpn60 nucleic acids from many microbes are available and can be used to design cpn60 probes (see, for example, GenBank Accession Nos. NC_{—}003366, NC_{—}000913, AL939121, NC_{—}002163, and NC_{—}003198; SEQ ID NOS:1-5, respectively). See also, U.S. Pat. No. 6,497,880, describing the sequences of Aspergillus fumigatus cpn60 and Candida glabrata cpn60. cpn60 nucleic acid sequences from other microbial species also are known to those of skill in the art, and can be used to detect and quantify cpn60-containing microbes in a sample.

The invention provides cpn60 probes that can be used to detect and quantify cpn60 nucleic acid molecules. As used herein, the term “cpn60 probes” refers to oligonucleotide probes that anneal to cpn60 nucleic acids, e.g., chromosomal cpn60 sequences. Probes that hybridize to a microbial cpn60 nucleic acid sequence (e.g., a Clostridium perfringens cpn60 sequence) can be designed using, for example, a computer program such as OLIGO (Molecular Biology Insights, Inc., Cascade, Colo.). Species-specific cpn60 probes can be designed to hybridize preferentially to cpn60 nucleotide sequences from a particular microbial species. As used herein, a “species-specific” cpn60 probe hybridizes preferentially to the cpn60 nucleic acid sequence of a particular microbial species, while a “universal” cpn60 probe can hybridize to cpn60 nucleic acid sequences from more than one species. Universal cpn60 probes can be designed to hybridize to a conserved target sequence found in the cpn60 nucleic acid sequence of multiple species, thus allowing for simultaneous detection of more than one (or all) species within a sample. Universal cpn60 probes also can be designed to hybridize to a cpn60 nucleotide sequence that is conserved but that contains polymorphisms or mutations, thereby allowing for differential detection of cpn60-containing species. Such differential detection can be based either on absolute hybridization of different probes corresponding to particular species, or differential melting temperatures between, for example, a universal probe and cpn60 nucleic acids from various species. The length of a cpn60 probe must be sufficient for sequence-specific hybridization to occur, but not so long that fidelity is reduced during synthesis of the probe. cpn60 probes used for in situ hybridization typically are about 15 to about 2000 nucleotides in length (e.g., 15, 20, 25, 30, 40, 50, 100, 200, 300, 400, 500, 750, 1000, 1500, or 2000 nucleotides in length).

In Situ Hybridization Assays

In situ hybridization methods (e.g., fluorescence in situ hybridization (FISH)) can be used to determine a microbial profile and quantify microbial species within a sample. In general, the in situ hybridization methods provided herein include the steps of fixing a biological or non-biological sample, hybridizing a cpn60 probe to target DNA contained within the fixed sample, washing to remove non-specific binding, detecting the hybridized probe, quantifying the amount of hybridized probe, and correlating the amount of hybridized probe to the amount of one or more microbial species within the sample.

FISH offers many advantages over radioactive and chromogenic methods for localizing and determining the relative abundance of specific nucleic acid sequences in cells, tissue, interphase nuclei and metaphase chromosomes. Not only are fluorescence techniques fast and precise, they allow for simultaneous analysis of multiple probes that may be spatially overlapping. Through use of appropriate optical filters, it is possible to distinguish four to five different fluorescent signals in a single sample using their excitation and emission properties alone. By using defined ratios of two fluorescent labels per probe (called COBRA for combined binary ratio labeling) in conjunction with highly discriminating optical filters and appropriate software, over 40 signals can be distinguished on the same sample.

Typically, cells (e.g., microbial cells) are harvested from a biological or non-biological sample using standard techniques. For example, cells can be harvested by centrifuging a sample and resuspending the pelleted cells in, for example, phosphate-buffered saline (PBS). After re-centrifuging the cell suspension to obtain a cell pellet, the cells can be fixed in a solution such as an acid alcohol solution, an acid acetone solution, or an aldehyde such as formaldehyde, paraformaldehyde, or glutaraldehyde. For example, a fixative containing methanol and glacial acetic acid in a 3:1 ratio, respectively, can be used as a fixative. A neutral buffered formalin solution also can be used (e.g., a solution containing approximately 1% to 10% of 37-40% formaldehyde in an aqueous solution of sodium phosphate). Slides containing the cells can be prepared by removing a majority of the fixative, leaving the concentrated cells suspended in only a portion of the solution.

The cell suspension is applied to slides such that the cells do not overlap on the slide. Cell density can be measured by a light or phase contrast microscope. For example, cells harvested from a 20 to 100 ml urine sample typically are resuspended in a final volume of about 100 to 200 μl of fixative. Three volumes of this suspension (e.g., 3, 10, and 30 μl), are then dropped into 6 mm wells of a slide. The cellularity (i.e., the density of cells) in these wells is then assessed with a phase contrast microscope. If the well containing the greatest volume of cell suspension does not have enough cells, the cell suspension can be concentrated and placed in another well.

Probes for in situ hybridization methods such as FISH are chosen for maximal sensitivity and specificity. Using a set of probes (e.g., two or more cpn60 probes) can provide greater sensitivity and specificity than the use of any one probe. cpn60 probes typically are about 30 to about 2×10³ nucleotides in length (e.g., 30, 40, 50, 75, 100, 200, 300, 400, 500, 750, 1000, 1500, or 2000 nucleotides in length). Longer probes can comprise smaller fragments of about 100 to about 500 nucleotides in length. Probes that hybridize with locus-specific DNA can be obtained commercially from, for example, Vysis, Inc. (Downers Grove, Ill.), Molecular Probes, Inc. (Eugene, Oreg.), or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via PCR. See, for example, Nath and Johnson, Biotechnic Histochem., 1998, 73(1):6-22, Wheeless et al., Cytometry, 1994, 17:319-326, and U.S. Pat. No. 5,491,224.

cpn60 may be differentially expressed in different microbial species exposed to different environmental conditions (e.g., different temperatures or pH). To maximize the accuracy of quantitation of cpn60 and thus microbial species, cpn60 probes can be designed to hybridize to chromosomal DNA without hybridizing to mRNA. For example, a cpn60 probe can be designed to hybridize to the non-coding DNA strand, and thus will not hybridize to the coding strand or to mRNA transcribed from the corresponding region. Alternatively, a cpn60 probe can be designed to hybridize to a cpn60 nucleotide sequence that is not within the mRNA sequence (e.g., a cpn60 promoter sequence). Such probes will hybridize only to chromosomal cpn60, and thus should result in a ratio of two probe molecules per microbial cell.

Typically, cpn60 probes for FISH are directly labeled with a fluorescent moiety (also referred to as a fluorophore), an organic molecule that fluoresces after absorbing light of lower wavelength/higher energy. The fluorescent moiety allows the probe to be visualized without a secondary detection molecule. After covalently attaching a fluorophore to a nucleotide, the nucleotide can be directly incorporated into a probe using standard techniques such as nick translation, random priming, and PCR labeling. Alternatively, deoxycytidine nucleotides within a probe can be transaminated with a linker. A fluorophore then can be covalently attached to the transaminated deoxycytidine nucleotides. See, U.S. Pat. No. 5,491,224.

When short oligonucleotide probes are used for FISH, a secondary detection method may be required to amplify the signal. By using a series of multiply labeled oligonucleotides that recognize adjacent sequences, however, oligonucleotide probes can be sufficiently sensitive to detect a single RNA transcript in situ. In addition, molecular beacons that are labeled with a fluorophore and a quencher can provide the sensitivity required to detect 10 molecules of RNA in a single cell in situ without the need for amplification.

When more than one probe is used, fluorescent moieties of different colors can be chosen such that each probe in the set can be distinctly visualized and quantitated. For example, a combination of the following fluorophores may be used: 7-amino-4-methylcoumarin-3-acetic acid (AMCA), Texas Red™ (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3a, 4a diaza-3-indacenepropionic acid, eosin-5-isothiocyanate, erythrosin-5-isothiocyanate, and Cascade™ blue acetylazide (Molecular Probes, Inc.). Probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine and quantitate the hybridization pattern of the probes.

Probes also can be indirectly labeled with biotin or digoxygenin, or labeled with radioactive isotopes such as ³²P and ³H, although secondary detection molecules or further processing then may be required to visualize the probes and quantify the amount of hybridization. For example, a probe indirectly labeled with biotin can be detected and quantitated using avidin conjugated to a detectable enzymatic marker such as alkaline phosphatase or horseradish peroxidase. Enzymatic markers can be detected and quantitated in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the probes and the chromosomal DNA contained within the cell each are denatured. Denaturation typically is performed by incubating in the presence of high pH, heat (e.g., temperatures from about 70° C. to about 95° C.), organic solvents such as formamide and tetraalkylammonium halides, or combinations thereof. For example, chromosomal DNA can be denatured by a combination of temperatures above 70° C. (e.g., about 73° C.) and a denaturation buffer containing 70% formamide and 2×SSC (0.3 M sodium chloride and 0.03 M sodium citrate). Denaturation conditions typically are established such that cell morphology is preserved. Probes can be denatured by heat (e.g., by heating to about 73° C. for about five minutes).

After removal of denaturing chemicals or conditions, probes are annealed to the chromosomal DNA under hybridizing conditions. “Hybridizing conditions” are conditions that facilitate annealing between a probe and target chromosomal DNA. Hybridization conditions vary, depending on the concentrations, base compositions, complexities, and lengths of the probes, as well as salt concentrations, temperatures, and length of incubation. The higher the concentration of probe, the higher the probability of forming a hybrid. For example, in situ hybridizations typically are performed in hybridization buffer containing 1-2×SSC, 50% formamide, and blocking DNA to suppress non-specific hybridization. In general, hybridization conditions, as described above, include temperatures of about 25° C. to about 55° C., and incubation times of about 0.5 hours to about 96 hours. More particularly, hybridization can be performed at about 32° C. to about 40° C. for about 2 to about 16 hours.

Non-specific binding of probes to DNA outside of the target region can be removed by a series of washes. The temperature and concentration of salt in each wash depend on the desired stringency. For example, for high stringency conditions, washes can be carried out at about 65° C. to about 80° C., using 0.2× to about 2×SSC, and about 0.1% to about 1% of a non-ionic detergent such as Nonidet P-40 (NP40). Stringency can be lowered by decreasing the temperature of the washes or by increasing the concentration of salt in the washes.

The amount of specifically-bound cpn60 probe can be quantified after removal of non-specific binding. The amount of bound probe then can be correlated to the amount(s) of various microbial species present in the sample. For example, the amount of fluorophore incorporated into a cpn60 probe can be known or determined, and this value in turn can be used to determine the amount of nucleic acid to which the probe binds. In conjunction with analysis of control samples (e.g., serially diluted samples) containing known numbers of microbial organisms, the number of microbial organisms in a biological or non-biological sample can be determined. When species-specific cpn60 probes are used, the amount of hybridization of each probe can be correlated to quantitate the amounts (or relative amounts) of the various species containing nucleic acid sequences to which the probes bind. In addition, the digital imaging capabilities of a charge-coupled device camera system can be used to quantify the hybridization signals of one or more fluorescently labeled cpn60 probes. The hybridization signal ratios can be calculated for different combinations of probes to determine the relative amounts of each microbial species recognized by the various cpn60 probes.

In some embodiments, a control probe also is hybridized to the nucleic acid in a sample, and the amount of hybridization of the control probe is compared to the amount of hybridization of the cpn60 probe. Control probes can be generated against, for example, microbial “housekeeping” genes, which typically are stably expressed reference genes that encode proteins with activities that are essential for the maintenance of cell function. Due to the similar and essential role of these genes for cell viability, it is generally assumed that these genes are expressed at similar levels in different species. If multiple species-specific cpn60 probes are used, the detected amount of specific hybridization of each cpn60 probe to the sample is compared to the detected amount of specific hybridization of the control probe to the sample, and a ratio is determined for each cpn60 probe. In this manner, the relative amounts of different microbial species in the sample can be determined.

cpn60 Polypeptide Markers

As used herein, a cpn60 polypeptide marker is a polypeptide that includes all or a portion of a cpn60 protein. As with cpn60 probes, a cpn60 polypeptide marker can be specific to a particular microbial species or can be universal. A species-specific cpn60 polypeptide marker is all or a portion of a given species' cpn60 protein. In the methods of the present invention, the probe or analytical method for detecting a marker should be capable of discriminating between a species-specific cpn60 polypeptide and all other cpn60 polypeptides, e.g., by mass in mass-spectrometry applications or by a particular epitope in an antibody assay. For example, one of skill in the art will recognize that antibodies, particularly monoclonal antibodies (mAb), can be obtained that recognize an epitope that is specific to a particular species' cpn60 protein. Accordingly, use of such specific antibodies in the methods described herein allows the differential detection of a particular species in a sample.

In other embodiments, a cpn60-specific polypeptide marker can be universal. For example, a “universal” cpn60 polypeptide marker can be a common structural (conformational) epitope in two or more cpn60 proteins. As described more fully below, antibodies, particularly polyclonal antibodies, raised against cpn60 proteins or polypeptides can be screened for cross-reactivity to common epitopes on cpn60 polypeptides from two or more microbes.

cpn60 Polypeptide-based Assays

The invention provides cpn60 polypeptide-based methods for determining a microbial profile (e.g., detecting and/or quantifying microbial species) within a biological or non-biological sample. A cpn60 protein or cpn60 polypeptide can be used as a universal target to determine the presence or absence of one or more microbes, and further can be used as a species-specific target and/or probe for the identification and quantification of specific microbes within a biological or non-biological sample. Such assays can be used on their own or in conjunction with other procedures (e.g., in situ hybridization-based assays).

In the assays of the invention, the presence or absence of a cpn60 polypeptide is detected and, in some embodiments, its level is measured. Methods of detecting and/or measuring the levels of a protein of interest in samples are known in the art. Many such methods employ antibodies (e.g., polyclonal antibodies or mAbs) that bind specifically to the protein of interest. Antibodies having specific binding affinities for a cpn60 protein or a cpn60 polypeptide can be produced using standard methods. As used herein, the terms “antibody” and “antibodies” include intact molecules as well as fragments thereof that are capable of binding to an epitopic determinant of a cpn60 polypeptide. The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). The terms “antibody” and “antibodies” include polyclonal antibodies, monoclonal antibodies, humanized or chimeric antibodies, single chain Fv antibody fragments, Fab fragments, and F(ab)₂ fragments.

Antibodies can be specific for a particular cpn60 polypeptide, e.g., the cpn60 protein of Clostridium perfringens. Alternatively, antibodies can be cross-reactive with two or more cpn60 polypeptides (e.g., can bind to cpn60 polypeptides from two or more species). For example, such antibodies can bind to common epitopes present in two or more cpn60 proteins or polypeptides. As used herein, antibodies with specificity for two or more cpn60 polypeptides are termed “universal” antibodies. In some embodiments, anti-cpn60 antibodies can bind to common epitopes present in all cpn60 polypeptides. Such antibodies thus may be able to detect the presence or absence of any microbe in a sample, and can optionally be used to determine the relative concentration or amount of the microbe.

In other embodiments, the identification and quantification of a particular microbe may be preferred. Accordingly, an antibody specific for a particular cpn60 polypeptide can be employed, either alone or in conjunction with a universal antibody; such antibodies are referred to herein as “species-specific” antibodies. Universal and species-specific antibodies can be employed simultaneously or in series. For example, a universal antibody may be used as a first screen to determine the presence or absence of a cpn60 polypeptide. Subsequently, a species-specific antibody, such as one specific for a cpn60 polypeptide of a particular microbe, e.g., Campylobacter jejuni, can be employed. In such assays, monoclonal antibodies may be particularly useful (e.g., sensitive) to identify cpn60 polypeptides of a particular microbe.

In general, a protein of interest (e.g., a cpn60 protein against which one wishes to prepare antibodies) is produced recombinantly, by chemical synthesis, or by purification of the native protein, and then used to immunize animals. As used herein, an intact cpn60 protein may be employed, or a cpn60 polypeptide may be employed, provided that the cpn60 polypeptide is capable of generating the desired immune response. See, for example, WO 200265129 for examples of epitopic sequences that bind to human antibodies against Chlamydia trachomatis; such epitopic sequences may be useful in generating antibodies against Chlamydia spp. for use in the present invention. See also U.S. Pat. No. 6,497,880, which sets forth nucleic acid sequences, amino acid sequences, expression vectors, purified proteins, antibodies, etc. specific to Aspergillus fumigatus and Candida glabrata. Purified Aspergillus fumigatus and Candida glabrata proteins, or proteolytically or synthetically generated fragments thereof, can be used to immunize animals to generate antibodies for use in the methods of the present invention. Finally, see WO 200257784, which discloses substantially purified Chlamydia hsp60 (cpn60) polypeptides. Such polypeptides also can be used to generate antibodies for use in the methods of the present invention.

As discussed previously, one may wish to prepare universal or species-specific antibodies to cpn60 proteins or polypeptides. A cpn60 polypeptide can be used to generate a universal antibody if, for example, it contains an epitope that is common to at least two cpn60 proteins, or, e.g., to all cpn60 proteins that one wishes to detect (e.g., the cpn60 proteins of the Campylobacter genera). Alternatively, a cpn60 protein or cpn60 polypeptide can be used to generate antibodies specific for a particular cpn60 protein or polypeptide present in a particular microbe, e.g., only Campylobacter jejuni.

Various host animals including, for example, rabbits, chickens, mice, guinea pigs, and rats, can be immunized by injection of the protein of interest. Adjuvants can be used to increase the immunological response depending on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin (KLH), and dinitrophenol. Polyclonal antibodies are heterogenous populations of antibody molecules that are specific for a particular antigen, which are contained in the sera of the immunized animals. Monoclonal antibodies, which are homogeneous populations of antibodies to a particular epitope contained within an antigen, can be prepared using standard hybridoma technology. In particular, monoclonal antibodies can be obtained by any technique that provides for the production of antibody molecules by continuous cell lines in culture such as described by Kohler et al. (1975) Nature 256:495, the human B-cell hybridoma technique (Kosbor et al. (1983) Immunology Today 4:72; Cote et al. (1983) Proc. Natl. Acad. Sci. USA 80:2026), and the EBV-hybridoma technique (Cole et al., “Monoclonal Antibodies and Cancer Therapy” Alan R. Liss, Inc., 1983, pp. 77-96). Such antibodies can be of any immunoglobulin class, including IgG, IgM, IgE, IgA, IgD, and any subclass thereof. A hybridoma producing monoclonal antibodies of the invention can be cultivated in vitro or in vivo.

A chimeric antibody is a molecule in which different portions are derived from different animal species, such as those having a variable region derived from a murine monoclonal antibody and a human immunoglobulin constant region. Chimeric antibodies can be produced using standard techniques.

Antibody fragments that have specific binding affinity for a cpn60 polypeptide also can be generated by known techniques. Such fragments include, but are not limited to, F(ab′)₂ fragments that can be produced by pepsin digestion of an antibody molecule, and Fab fragments that can be generated by reducing the disulfide bridges of F(ab′)₂ fragments. Alternatively, Fab expression libraries can be constructed. See, for example, Huse et al. (1989) Science 246:1275. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques. See, for example, U.S. Pat. No. 4,946,778.

Once produced, antibodies or fragments thereof can be tested for recognition of a cpn60 protein or cpn60 polypeptide by standard immunoassay methods including, for example, ELISA techniques, countercurrent immuno-electrophoresis (CIEP), radioimmunoassays (RIA), radioimmunoprecipitations, dot blots, inhibition or competition assays, sandwich assays, immunostick (dipstick) assays, immunochromatographic assays, immunofiltration assays, latex beat agglutination assays, immunofluoroescent assays, and/or biosensor assays. See, Short Protocols in Molecular Biology, Chapter 11, Green Publishing Associates and John Wiley & Sons, edited by Ausubel et al., 1992; Antibodies: A Laboratory Manual, Harlow and Lane (eds.), Cold Spring Harbor Laboratory Press, 1988; and U.S. Pat. Nos. 4,376,110; 4,486,530; and 6,497,880. Antibodies and antibody fragments also can be tested for their ability to react universally (e.g., with two or more cpn60 proteins or cpn60 polypeptides, such as the cpn60 proteins from a bacterial genera such as Clostridium), or specifically with a particular cpn60 protein (e.g., the cpn60 protein of Clostridium perfringens).

In antibody assays, the antibody itself or a secondary antibody that binds to it can be detectably labeled. Alternatively, an antibody can be conjugated with biotin, and detectably labeled avidin (a protein that binds to biotin) can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. The methods described below for detecting a cpn60 polypeptide in a liquid sample also can be used to detect a cpn60 polypeptide in cell lysates.

Methods for detecting a cpn60 polypeptide in a liquid sample generally involve contacting a sample of interest with an antibody that binds to a cpn60 polypeptide and testing for binding of the antibody to a component of the sample. In such assays the antibody need not be detectably labeled and can be used without a second antibody that binds to a cpn60 polynucleotide. For example, an antibody specific for a cpn60 polynucleotide may be bound to an appropriate solid substrate and then exposed to the sample. Binding of a cpn60 polypeptide to an antibody on the solid substrate can be detected by exploiting the phenomenon of surface plasmon resonance, which results in a change in the intensity of surface plasmon resonance upon binding that can be detected qualitatively or quantitatively by an appropriate instrument, e.g., a Biacore apparatus (Biacore International AB, Rapsgatan, Sweden).

Assays for detection of a cpn60 polypeptide in a liquid sample also can involve the use of, for example: (a) a single, detectably labeled antibody specific for a cpn60 polypeptide; (b) an unlabeled antibody that is specific for a cpn60 polypeptide and a detectably labeled secondary antibody that either does or does not recognize cpn60; or (c) a biotinylated antibody specific for a cpn60 polypeptide and detectably labeled avidin. In addition, combinations of these approaches (including “multi-layer” assays) familiar to those in the art can be used to enhance the sensitivity of assays. In these assays, a sample or an aliquot of a sample suspected of containing a microbe can be immobilized on a solid substrate, such as a nylon or nitrocellulose membrane, by, for example, “spotting” an aliquot of a liquid sample or by blotting of an electrophoretic gel on which the sample or an aliquot of the sample has been subjected to electrophoretic separation. The presence or amount of cpn60 polypeptide on the solid substrate then can be assayed using any of the above-described forms of anti-cpn60 polypeptide specific antibodies and, where required, appropriate detectably labeled secondary antibodies or avidin.

The invention also features “sandwich” assays. In sandwich assays, rather than immobilizing samples on solid substrates by the methods described above, a “capture” antibody (polyclonal or mAb) specific for a cpn60 polypeptide is conjugated to the solid substrate by any of a variety of methods known in the art. A sample is then passed over the solid substrate, and cpn60 polypeptides that may be present in the sample can interact with the capture antibody and thus become coupled to the solid substrate. The presence or amount of cpn60 polypeptide bound to the conjugated capture antibody then can be assayed using a “detection” antibody specific for a cpn60 polypeptide, using methods essentially the same as those described above for using a single antibody specific for a cpn60 polypeptide. It is understood that in such sandwich assays, the capture antibody should not bind to the same epitope (or range of epitopes in the case of a polyclonal antibody) as the detection antibody. Thus, if a mAb is used as a capture antibody, the detection antibody can be either (a) another mAb that binds to a cpn60 epitope that is either completely physically separated from or only partially overlaps the epitope to which the capture mAb binds; (b) a polyclonal antibody that binds to cpn60 epitopes other than or in addition to that to which the capture mAb binds; or (c) an antibody that does not recognize cpn60. On the other hand, if a polyclonal antibody is used as a capture antibody, the detection antibody can be either (a) a mAb that binds to a cpn60 epitope to that is either completely physically separated from or partially overlaps any of the epitopes to which the capture polyclonal antibody binds; (b) a polyclonal antibody that binds to cpn60 epitopes other than or in addition to that to which the capture polyclonal antibody binds, or (c) an antibody that does not bind to cpn60. Assays that involve the use of capture and detection antibodies include sandwich ELISA assays, sandwich Western blotting assays, and sandwich immunomagnetic detection assays.

Suitable solid substrates to which capture antibodies can be bound include, without limitation, the plastic bottoms and sides of wells of microtiter plates, membranes such as nylon or nitrocellulose membranes, and polymeric (e.g., agarose, cellulose, or polyacrylamide) beads or particles. It is noted that antibodies bound to such beads or particles also can be used for immunoaffinity purification of cpn60 polypeptides. Immunostick formats can employ a solid phase such as, without limitation, a polystyrene paddle or dispstick.

Methods for detecting and/or quantifying a detectable label depend on the nature of the label. Suitable labels include, without limitation, radionuclides (e.g., ¹²⁵I, ¹³¹I, ³⁵S, ³H, ³²P, ³³P, and ¹⁴C), fluorescent moieties (e.g., fluorescein, rhodamine, and phycoerythrin), luminescent moieties (e.g., Qdot™ nanoparticles supplied by the Quantum Dot Corporation, Palo Alto, Calif.), compounds that absorb light of a defined wavelength, and enzymes (e.g., alkaline phosphatase and horseradish peroxidase). The products of reactions catalyzed by such enzymes can be, without limitation, fluorescent, luminescent, or radioactive, or they may absorb visible or ultraviolet light. Detectors of the various types of labels disclosed herein include, without limitation, x-ray film, radioactivity counters, scintillation counters, spectrophotometers, colorimeters, fluorometers, luminometers, and densitometers.

As for the in situ hybridization assays described herein, the amount of specifically-bound anti-cpn60 antibody can be quantified. The amount of bound antibody then can be correlated to the amount(s) of various microbial species present in the sample. For example, the amount of fluorophore incorporated into an anti-cpn60 antibody can be known or determined, and this value in turn can be used to determine the amount of cpn60 polypeptide to which the antibody is bound. In conjunction with analysis of control samples (e.g., serially diluted samples) containing known numbers of microbial organisms, the number of microbial organisms in a biological or non-biological sample can be determined. When two or more species-specific anti-cpn60 antibodies (e.g., species-specific mAbs against cpn60 from more than one microbial organism) are used, the amount of binding of each antibody can be correlated to the amounts (or relative amounts) of the various species containing polypeptides recognized by the antibodies. In addition, the digital imaging capabilities of a charge-coupled device camera system can be used to quantify the signals of one or more fluorescently labeled anti-cpn60 antibodies. The signal ratios can be calculated for different combinations of antibodies to determine the relative amounts of each microbial species recognized by the various species-specific anti-cpn60 antibodies.

The methods provided herein can employ a control sample. In assays to detect the presence or absence of a microbe, the concentration of a cpn60 polypeptide in, for example, a food sample suspected of being contaminated, or at risk of being contaminated, with a microbe can be compared to a control sample, e.g., a food sample known not to be infected. The control sample can be taken from the same environment, e.g., in a different location known to be uncontaminated, or can be a control sample taken from a different environment. Alternatively, a control sample can be taken from the same environment but at an earlier or later time-point when the location was known to be uncontaminated. A significantly higher concentration of cpn60 polypeptide in the suspect sample relative to the control sample would indicate the presence of a microbe.

It is understood that, while the above descriptions of diagnostic assays may refer to assays on food samples or bodily fluid samples, the assays also can be carried out on any of the other fluid or solubilized samples listed herein, such as water samples or buffer samples (e.g., buffer used to extract a sample from a fomite).

The present invention also contemplates the use of other analytical techniques for detecting cpn60 polypeptides. Recent analytical instrumentation and methodology advances that have arisen in the context of proteomics research are applicable in methods of the present invention. See, generally, Jungblut (2001) Microbes & Infection 3:831-840; MacBeath and Schreiber (2000) Science 289:1760-1763; Madoz-Gdrpide, Wang, and Misek (2001) Proteomics 1:1279-1287; Patterson (2000) Physiological Genomics 2:59-65; and Schevchenko et al. (2000) Analytical Chemistry 72:2132-2141.

Mass-spectrophotometric techniques have increasingly been used to detect and identify proteins and protein fragments at low levels, e.g., fmol or pmol. Mass spectrometry has become a major analytical tool for protein and proteomics research because of advancements in the instrumentation used for biomolecular ionization, electrospray ionization (ESI), and matrix-assisted laser desorption-ionization (MALDI). MALDI usually is combined with a time-of-flight (TOF) mass analyzer. Typically, about 0.5 μl of a biological or non-biological sample that contains about 1-10 pmol of protein or peptide is mixed with an equal volume of a saturated matrix solution and allowed to dry, resulting in the co-crystallization of the analyte with the matrix. Useful matrix compounds include, for example, sinapic acid and α-hydroxycinnamic acid. The cocrystallized material on the target plate is irradiated with a nitrogen laser pulse, e.g., at a wavelength of 337 nm, to volatilize and ionize the protein or peptide molecules. A strong acceleration field is switched on, and the ionized molecules move down the flight tube to a detector. The amount of time required to reach the detector is related to the mass-to-charge ratio. Proteolytic mass mapping and tandem mass spectrometry, when combined with searches of protein and protein fragment databases, also can be employed to detect and identify cpn60 polypeptides. See, for example, Downard (2000) J. Mass. Spectrom. 35:493-503.

Biomolecular interaction analysis mass spectrometry (BIA-MS) is a technique suitable for detecting interactions between cpn60 polypeptides and cpn60 antibodies. This technology detects molecules bound to a ligand that is covalently attached to a surface. As the density of biomaterial on the surface increases, changes occur in the refractive index at the solution or surface interface. This change in the refractive index is detected by varying the angle or wavelength at which the incident light is absorbed at the surface. The difference in the angle or wavelength is proportional to the amount of material bound on the surface, giving rise to a signal that is termed surface plasmon resonance (SPR), as discussed previously. See, for example, Nelson et al. (1999) Analytical Chemistry 71:2858-2865; and Nedelkov and Nelson (2001) Biosensors and Bioelectronics 16:1071-1078.

The SPR biosensing technology has been combined with MALDI-TOF mass spectrometry for desorption and identification of biomolecules. In a chip-based approach to BIA-MS, a ligand, e.g., a cpn60 antibody, is covalently immobilized on the surface of a chip. A tryptic digest of solubilized proteins from a sample is routed over the chip, and the relevant peptides, e.g., cpn60 polypeptides, can bind to the ligand. After a washing step, the eluted peptides are analyzed by MALDI-TOF mass spectrometry. The system may be a fully automated process and is applicable to detecting and characterizing proteins present in complex biological fluids and cell extracts at low- to sub-femtomolar levels.

Mass spectrometers useful for such applications are available from Applied Biosystems (Foster City, Calif.); Bruker Daltronics (Billerica, Mass.) and Amersham Pharmacia (Sunnyvale, Calif.). Software for quantifying polypeptides subjected to mass spectrometry can be obtained commercially from, for example, Thermo Finnigan (San Jose, Calif.).

Other suitable techniques for use in the present invention include “Multidimensional Protein Identification Technologies (MUDPIT).” Cells are fractionally solubilized and digested, e.g., sequentially with endoproteinase Lys-C and immobilized trypsin. The samples are then subjected to MUDPIT, which involves a sequential separation of the peptide fragments by on-line biphasic microcapillary chromatography (e.g., strong ion exchange and C-18 separation), followed by tandem mass spectrometry (MS-MS). See, for example, Washburn, Wolter, and Yates (2001) Nature Biotechnology 19:242-247.

Sampling devices can be employed for general quantification of cpn60 polypeptides in biological and non-biological samples. For example, a sampling device can have (a) a porous or semipermeable compartment containing a known amount of a particular microbial species; and (b) a second compartment for collecting a biological sample (e.g., a sample of fecal matter). The sampling device can be inserted into the fecal sample and incubated there for a suitable length of time (e.g., a length of time that is long enough for the compartments to equilibrate to the temperature and general environment of the fecal sample, but shorter than the doubling time of the microbial species contained within the first compartment). The device then can be withdrawn from the sample, and samples in both compartments can be analyzed for the level of cpn60 polypeptides. By comparing the relative amount of cpn60 polypeptides in each compartment, the amount of microbes in the second compartment can be determined. In some embodiments, a sampling device can contain a plurality of semi-permeable or porous compartments, each containing a known amount of a different microbial species, or each containing a different amount of a single microbial species.

Due to differential expression of cpn60 by different organisms in response to heat or other stress, certain cpn60 polypeptide-based methods may be most useful for determining microbial profiles that simply identify microbial species present within a sample. Such methods can include, for example, contacting a biological or non-biological sample with a plurality of species-specific anti-cpn60 antibodies (e.g., a cocktail containing a plurality of such antibodies) that are detectably labeled with different moieties (e.g., different fluorophores). Detection of each particular label indicates the presence of a corresponding particular microbial species in the sample. The resulting microbial profile is not quantitative, but is useful to identify microbes present within a sample.

Articles of Manufacture

The invention also provides articles of manufacture. Articles of manufacture can include at least one cpn60 oligonucleotide probe, as well as instructions for using the cpn60 probe to quantify the amount of one or more microbial organisms in a biological or non-biological sample. The cpn60 probe can be labeled (e.g., with a fluorescent moiety). Suitable cpn60 oligonucleotide probes include those that are complementary to highly conserved regions of cpn60. Such universal cpn60 probes can be used to detect and quantify multiple species of microorganisms. Suitable cpn60 oligonucleotide probes also include those that are complementary to species-specific cpn60 sequences, and thus result in detection and quantification only if a particular species is present in the sample. Articles of manufacture provided herein further can include additional components for carrying out in situ hybridization reactions, for example, slides or other solid supports.

The invention also provides articles of manufacture including at least one cpn60 antibody, as well as instructions for using the antibody or antibodies to detect and quantify the presence of a microbe, and optionally to evaluate a microbial profile, in a biological or non-biological sample.

In one embodiment, one or more cpn60 antibodies are attached to a microarray (e.g., a 96-microwell plate). For example, a microarray fornat can include a variety of universal and specific cpn60 capture antibodies; the universal and specific antibodies may each be located at a different well location. The article of manufacture also can include appropriate detection antibodies, if necessary, and appropriate reagents for detection of binding of a cpn60 polypeptide to one or more capture antibodies (e.g., enzymes, substrates, buffers, and controls).

In another embodiment, an article of manufacture can include one or more cpn60 antibodies attached to a dipstick. Such dipsticks can be used, for example, to detect cpn60 polypeptides in a liquid sample. The invention further provides sampling devices such as those described above.

It will be appreciated by those of ordinary skill in the art that different articles of manufacture can be provided to evaluate microbial profiles of different types of samples (e.g., biological samples from different types of animals). For example, the microbial profile of the pig GIT has a different community of microbes than that of poultry. Therefore, an article of manufacture designed to evaluate the microbial profile of, for example, the pig GIT may have a different set of controls or a different set of species-specific hybridization probes than that designed for poultry. Alternatively, a more generalized article of manufacture can be used to evaluate the microbial profiles of a number of different animal species.

The invention will be further described in the following examples, which do not limit the scope of the invention described in the claims.

### EXAMPLES

### Example 1—Dipstick ELISA Assay for Streptococcus

A polystyrene dipstick containing two horizontal bands is constructed: one band consists of broadly reactive, polyclonal capture antibodies against cpn60 proteins from Streptococcus spp., while the other band is an internal control consisting of horseradish peroxidase. The assay is performed by making serial dilutions (1:2, 1:5, 1:10, etc.) of a liquid sample taken from a high risk environment (e.g., a urine sample or a blood sample) directly into a detection reagent and incubating a wetted dipstick in these dilutions for 5 minutes, and then adding an indicator to detect binding of cpn60 proteins to the capture (and detection) antibodies. The detection reagent includes a suitable buffer and secondary cpn60 Streptococcus detection antibodies labeled with horseradish peroxidase. The indicator is a chromogenic horseradish peroxidase substrate, such as 2,2′-AZINO-bis 3-ethylbenziazoline-6-sulfonic acid, or ABTS. ABTS is considered a safe, sensitive substrate for horseradish peroxidase that produces a blue-green color upon enzymatic activity that can be quantitated at 405-410 nm. At the end of the incubation and indicator steps, the dipstick is rinsed with water (e.g., deionized water) and examined for staining of the antibody band by visual inspection. Staining of the antibody band reveals the presence of Streptococcus spp. in the sample. The internal control band provides a check on the integrity of the detection reagent.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for quantifying the amount of one or more microbial species in a biological or non-biological sample, said method comprising:
(a) contacting said sample in situ with at least one labeled cpn60 probe under conditions wherein said probe preferentially hybridizes to cpn60 nucleic acids, if present, in said sample; and
(b) quantifying the amount of said probe hybridized to said sample, wherein the amount of said hybridized probe is correlated with the amount of said microbial species in said sample.

2. The method of claim 1, wherein said at least one cpn60 probe is labeled with a fluorescent moiety and said hybridization is fluorescent in situ hybridization.

3. The method of claim 2, wherein said fluorescent moiety is selected from the group consisting of 7-amino-4-methylcoumarin-3-acetic acid, 5-carboxy-X-rhodamine, 6-carboxy-X-rhodamine, lissamine rhodamine B, 5-carboxyfluorescein, 6-carboxyfluorescein, fluorescein-5-isothiocyanate, 7-diethylaminocoumarin-3-carboxylic acid, tetramethylrhodamine-5-isothiocyanate, tetramethylrhodamine-6-isothiocyanate, 5-carboxytetramethylrhodamine, 6-carboxytetramethylrhodamine, 7-hydroxycoumarin-3-carboxylic acid, 6-[fluorescein 5-carboxamido]hexanoic acid, 6-[fluorescein 6-carboxamido]hexanoic acid, N-(4,4-difluoro-5,7-dimethyl-4-bora-3a ,4a diaza-3-indacenepropionic acid, eosin-5-isothiocyanate, and erythrosin-5-isothiocyanate.

4. The method of claim 1, wherein said correlation employs a standard curve of hybridization to cpn60 nucleic acids from known amounts of microbial species.

5. The method of claim 1, wherein said sample is contacted with at least two labeled cpn60 probes.

6. The method of claim 5, wherein said at least two labeled cpn60 probes are labeled with different fluorescent moieties.

7. The method of claim 1, wherein said sample is selected from the group consisting of a biological tissue, a biological fluid, a biological elimination product, a water sample, a soil sample, and a swab from an inanimate object.

8. The method of claim 1, wherein said one or more microbial species belong to genera selected from the group consisting of Escherichia, Salmonella, Campylobacter, Staphylococcus, Clostridium, Pseudomonas, Bifidobacterium, Bacillus, Enterococcus, Acanthamoeba, Cryptosporidium, Tetrahymena, Aspergillus, Candida, and Saccharomyces.

9. A method for quantifying the amount of one or more microbial species in a biological or non-biological sample, said method comprising detecting and/or quantifying the amount of cpn60 polypeptide from said microbial species, if present, in said sample, wherein the amount of said cpn60 polypeptide is correlated with the amount of said microbial species in said sample.

10. The method of claim 9, wherein said detecting and/or quantifying comprises contacting said sample with an anti-cpn60 antibody.

11. The method of claim 10, wherein said anti-cpn60 antibody is detectably labeled.

12. The method of claim 10, wherein said anti-cpn60 antibody is a monoclonal antibody.

13. The method of claim 10, wherein said anti-cpn60 antibody is a polyclonal antibody.

14. The method of claim 10, wherein said detecting and/or quantifying further comprises contacting said sample with a second antibody.

15. The method of claim 14, wherein said second antibody is an anti-cpn60 antibody.

16. The method of claim 14, wherein said second antibody does not bind to cpn60.

17. The method of claim 14, wherein said detecting and/or quantifying comprises a “sandwich” assay.

18. The method of claim 14, wherein said detecting and/or quantifying comprises an enzyme linked immunosorbent assay.

19. The method of claim 9, wherein said sample is selected from the group consisting of a biological tissue, a biological fluid, a biological elimination product, a water sample, a soil sample, and a swab from an inanimate object.

20. The method of claim 9, wherein said one or more microbial species belong to genera selected from the group consisting of Escherichia, Salmonella, Campylobacter, Staphylococcus, Clostridium, Pseudomonas, Bifidobacterium, Bacillus, Enterococcus, Acanthamoeba, Cryptosporidium, Tetrahymena, Aspergillus, Candida, and Saccharomyces.

21. A method for identifying one or more microbial species in a biological or non-biological sample, said method comprising detecting cpn60 polypeptides from said one or more microbial species, if present, in said sample.

22. The method of claim 21, wherein said detecting comprises contacting said sample with an anti-cpn60 antibody.

23. The method of claim 22, wherein said anti-cpn60 antibody is detectably labeled.

24. The method of claim 22, wherein said anti-cpn60 antibody is a monoclonal antibody.

25. The method of claim 22, wherein said anti-cpn60 antibody is a polyclonal antibody.

26. The method of claim 22, wherein said detecting and/or quantifying further comprises contacting said sample with a second antibody.

27. The method of claim 26, wherein said second antibody is an anti-cpn60 antibody.

28. The method of claim 26, wherein said second antibody does not bind to cpn60.

29. The method of claim 26, wherein said detecting comprises a “sandwich” assay.

30. The method of claim 26, wherein said detecting comprises an enzyme linked immunosorbent assay.

31. The method of claim 21, wherein said sample is selected from the group consisting of a biological tissue, a biological fluid, a biological elimination product, a water sample, a soil sample, and a swab from an inanimate object.

32. The method of claim 21, wherein said one or more microbial species belong to genera selected from the group consisting of Escherichia, Salmonella, Campylobacter, Staphylococcus, Clostridium, Pseudomonas, Bifidobacterium, Bacillus, Enterococcus, Acanthamoeba, Cryptosporidium, Tetrahymena, Aspergillus, Candida, and Saccharomyces.
